(19) United States
(12) Patent Application Publication
(10) Pub. No.: US 2013/0131490 A1
(43) Pub. Date: May 23, 2013

(54) System and Method for Determining The Presence of a Neurodegenerative Disease Using Magnetic Resonance Elastography

(71) Applicants: John Huston, III, Rochester, MN (US); Clifford R. Jack, JR., Rochester, MN (US); Kevin J. Glaser, Rochester, MN (US); Armando Manduca, Rochester, MN (US); Joel P. Felmlee, Rochester, MN (US); Richard L. Ehman, Rochester, MN (US); Matthew C. Murphy, Rochester, MN (US)

(72) Inventors: John Huston, III, Rochester, MN (US); Clifford R. Jack, JR., Rochester, MN (US); Kevin J. Glaser, Rochester, MN (US); Armando Manduca, Rochester, MN (US); Joel P. Felmlee, Rochester, MN (US); Richard L. Ehman, Rochester, MN (US); Matthew C. Murphy, Rochester, MN (US)

(21) Appl. No.: 13/453,070

(22) Filed: Apr. 23, 2012

Related U.S. Application Data
(60) Provisional application No. 61/478,280, filed on Apr. 22, 2011.

(51) Int. Cl. A61B 5/055

(52) U.S. Cl. CPC A61B 5/055
USPC 600/410

(57) ABSTRACT

A system and method for analyzing a subject using a magnetic resonance imaging (MRI) system is provided. The technique includes positioning a subject within the MRI system and coupling a driver to the subject to impart vibrational energy to the subject. The technique further includes using the MRI system and in coordination with operation of the driver, acquiring medical imaging data from the subject's brain and deriving stiffness information of the subject's brain from the medical imaging data. A report, such as an image, can be provided indicating the stiffness information of the subject's brain relative to baseline stiffness information to indicate a status of the subject with respect to a neurodegenerative disease.

### CROSS REFERENCE

This application is based on, claims the benefit of, and incorporates herein by reference U.S. Provisional Application Ser. No. 61/478,280 filed Apr. 22, 2011, and entitled “SYSTEM AND METHOD FOR DETERMINING THE PRESENCE OF A NEURODEGENERATIVE DISEASE USING MAGNETIC RESONANCE ELASTOGRAPHY.”

### STATEMENT REGARDING FEDERALLY SPONSORED RESEARCH

This invention was made with government support under EB001981 awarded by the National Institutes of Health. The government has certain rights in this invention.

### BACKGROUND OF THE INVENTION

The field of the invention relates to magnetic resonance imaging methods and systems. More particularly, the invention relates to systems and methods for performing magnetic resonance elastography (MRE) to provide clinical information relating to potential neurodegenerative diseases and, in particular, Alzheimer's disease.

Alzheimer's disease (AD) is characterized clinically by the progressive impairment of specific cognitive functions including memory, language, motor skills and perception. Pathologically, AD demonstrates the accumulation of extracellular amyloid plaques, intracellular neurofibrillary tangles and neurodegeneration. Due to changing demographics, the disease is expected to dramatically increase in prevalence in the US population, growing from 4.5 million people with AD today to an estimated 13.2 million by 2050. Improved treatment could mitigate the impact of the predicted increase in AD prevalence, and that treatment would be aided by earlier and more sensitive detection of the disease.

Clinicians have many diagnostic tools at their disposal that enable detection and localization of diseased tissues. These include x-ray systems that measure and produce images indicative of the x-ray attenuation of the tissues and ultrasound systems that detect and produce images indicative of tissue echogenicity and the boundaries between structures of differing acoustic properties. Nuclear medicine produces images indicative of those tissues which absorb tracers injected into the patient, as do PET scanners and SPECT scanners. And finally, magnetic resonance imaging (MRI) systems produce images indicative of the magnetic properties of tissues. It is fortuitous that many diseased tissues are detected by the physical properties measured by these imaging modalities, but it should not be surprising that many diseases go undetected. In particular, neurodegenerative diseases, such as AD can be particularly difficult to detect using non-invasive medical imaging methods.

It has been found that MR imaging can be enhanced when an oscillating stress is applied to the object being imaged in a method called MR elastography (MRE). MRE is gaining wider clinical applicability due to its ability to noninvasively and quantitatively measure tissue stiffness. MRE is a three-step process beginning with the induction of shear waves in the tissue to be examined via an external source of vibration. Second, the shear waves are imaged with a phase-contrast MRI pulse sequence with motion-encoding gradients synchronized with the applied vibration. Finally, the images of the wave motion are inverted to calculate the tissue stiffness. MRE is analogous to manual palpation, which has a long history in the practice of medicine as a clinical diagnostic tool for examining tissues such as the breast and thyroid for focal and diffuse diseases. In fact, MRE of the liver has already matured to a point where it is replacing needle biopsies for the diagnosis of fibrosis and cirrhosis in a growing number of clinical practices.

The method requires that the oscillating stress produce shear waves that propagate through the organ, or tissues to be imaged. These shear waves alter the phase of the MR signals, and from this the mechanical properties of the subject can be determined. In many applications, the production of shear waves in the tissues is merely a matter of physically vibrating the surface of the subject with an electromechanical device such as that disclosed in U.S. Pat. No. 5,592,085. For example, shear waves may be produced in the breast by placing the breast in direct contact with the oscillatory device. Also, with organs like the liver that are difficult to directly palpate, shear waves can be produced indirectly within the tissue by applying the oscillatory force to the exterior surface of the body and allowing the waves to propagate into the organ. Performing MRE of the brain presents additional unique technical challenges, including the introduction of shear waves through the bony calvarium, as well as performing efficient sampling and processing of a 3D displacement field.

Even with all of these and other diagnostic resources available to the clinician, the early detection of clear indicators of and the ultimate diagnosis of AD remains a substantial clinical challenge. For example, traditional MRI and molecular imaging methods have been used in an attempt to identify beta-amyloid plaques that may be indicative of AD. However, these techniques have found limited clinical adoption due to the need to accurately derive distinguishable contrast from the beta-amyloid plaques and/or quantify or qualify any detected beta-amyloid plaques in a manner that accurately translates to diagnosis or treatment of AD.

Therefore, it would be desirable to have a system and method for improving the detection of potential biomarkers for AD and to, ultimately, improve the clinical ability to diagnosis AD.

### SUMMARY OF THE INVENTION

The present invention overcomes the aforementioned drawback by providing a new biomarker for the detection of Alzheimer's disease (AD) and, thereby, improving diagnosis of AD. Specifically, the present invention provides a system and method for measuring the effect of brain amyloid on brain stiffness and for correlating measured changes in biological stiffness to a clinical diagnosis of AD. Furthermore, the present invention provides a system and method for accurately identifying and tracking the progression of AD over time to thereby provide valuable clinical information for the understanding and treatment of AD.

In accordance with one aspect of the present invention, a method for analyzing a subject using a magnetic resonance imaging (MRI) system includes positioning a subject within the MRI system and coupling a driver to the subject to impart vibrational energy to the subject. The technique further includes using the MRI system and in coordination with operation of the driver, acquiring medical imaging data from the subject's brain and deriving stiffness information of the subject's brain from the medical imaging data. A report, such as an image, can be provided indicating the stiffness information of the subject's brain relative to baseline stiffness information to indicate the status of the subject with respect to a neurodegenerative disease.

In accordance with another aspect of the invention, a magnetic resonance imaging (MRI) system is disclosed that includes a magnet system configured to generate a polarizing magnetic field about at least a portion of a subject and a plurality of gradient coils configured to apply a gradient field to the polarizing magnetic field. The system also includes a radio frequency (RF) system configured to apply an excitation field to the subject and acquire MR image data therefrom and a driver system configured to deliver an oscillatory stress to the subject to, thereby, direct a shear wave through the subject. The system includes a computer system programmed to control operation of the gradient coils and the driver system to coordinate characteristics of the oscillatory stress with application of the gradient field and control operation of the RF system to acquire medical imaging data from the subject's brain. The computer system is further programmed to derive stiffness information of the subject's brain from the medical imaging data and generate an image of the subject's brain from the medical imaging data indicating a status of the subject with respect to a neurodegenerative disease, based on the stiffness information.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of a magnetic resonance imaging (“MRI”) system that employs the present invention.

FIG. 2 is a graphic representation of an exemplary magnetic resonance elastography (“MRE”) pulse sequence employed by the MRI system of FIG. 1.

FIG. 3 is a flow chart setting forth the steps of an exemplary method in accordance with the present invention.

FIG. 4 is a report including a set of images for comparison including anatomical images, wave images, and elastograms.

FIG. 5 is a graph of results where each marker represents the median stiffness for a separate MRE exam and illustrates a tight distribution of stiffness measurements, all with a coefficient of variation less than 3.1%.

FIG. 6 is a graph of median stiffness for each volunteer in a study where lines indicate the median stiffness of each group.

### DETAILED DESCRIPTION

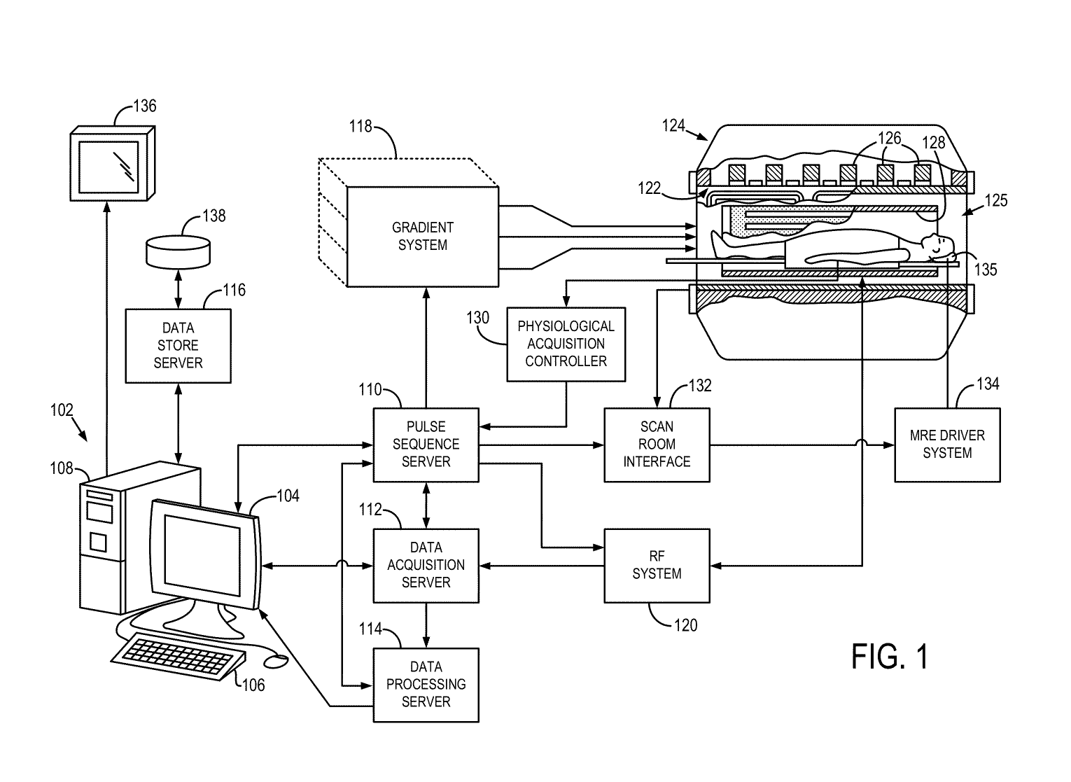

Referring to FIG. 1, the present invention is employed in a magnetic resonance imaging (“MRI”) system 100. The MRI system 100 includes a workstation 102 having a display 104 and a keyboard 106. The workstation 102 includes a processor 108, such as a commercially available programmable machine running a commercially available operating system. The workstation 102 provides the operator interface that enables scan prescriptions to be entered into the MRI system 100. The workstation 102 is coupled to four servers: a pulse sequence server 110; a data acquisition server 112; a data processing server 114, and a data store server 116. The workstation 102 and each server 110, 112, 114 and 116 are connected to communicate with each other.

The pulse sequence server 110 functions in response to instructions downloaded from the workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 extending about a bore 125 formed there through and includes a polarizing magnet 126 and a whole-body RF coil 128.

RF excitation waveforms are applied to the RF coil 128, or a separate local coil (not shown in FIG. 1), by the RF system 120 to perform the prescribed magnetic resonance pulse sequence. Responsive MR signals detected by the RF coil 128, or a separate local coil (not shown in FIG. 1), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays.

The RF system 120 also includes one or more RF receiver channels. Each RF receiver channel includes an RF amplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} Eqn. (1);

and the phase of the received MR signal may also be determined:

$$φ = {tan}^{- 1} ( \frac{Q}{I} ) . \qquad Eqn . ( 2 )$$

The pulse sequence server 110 also optionally receives patient data from a physiological acquisition controller 130. The controller 130 receives signals from a number of different sensors connected to the patient, such as electrocardiograph (“ECG”) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It may also be through the scan room interface circuit 132 that a MRE driver system 134 is coupled to the pulse sequence server 110 to, as will be described, coordinate operation of the an MRE driver 135, with the MRI system 100 to perform an MRE process.

A variety of MRE driver systems, including active and passive driver systems, are known. It is contemplated that, in accordance with the present invention, the MRE driver 135 may include an ergonomic flexible driver that provides desired mechanical coupling by conforming to the head of the subject to thereby generate a uniform and reproducible shear wave field. The vibration generated by the MRE driver 135 is designed to be well tolerated, even in AD patients who displayed moderate disorientation. Shear waves are introduced into the brain through, for example, the illustrated pillow-like driver 135 using, for example, a pneumatic actuator forming part of the MRE driver system 134. The active component of the actuator is typically located outside of the scan room, and may be formed of a waveform generator, an amplifier, and an acoustic speaker. The passive pillow-like driver 135 may be formed of a soft, inelastic, fabric cover over a porous, springy, mesh measuring 15×9×1.5 cm. The soft vibration source, as illustrated, may be placed under the subject's head within, for example, an 8 channel receive-only head coil. The active and passive driver components are connected, for example, by a 24-foot long, 0.75-inch diameter flexible tube from the active driver terminating in a 0.5-inch diameter, 1.5-foot long tube integrated into the passive driver.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110.

The data processing server 114 receives MR data from the data acquisition server 112 and processes it in accordance with instructions downloaded from the workstation 102. Such processing may include, for example: transformation of MRE wave images into elastograms; Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

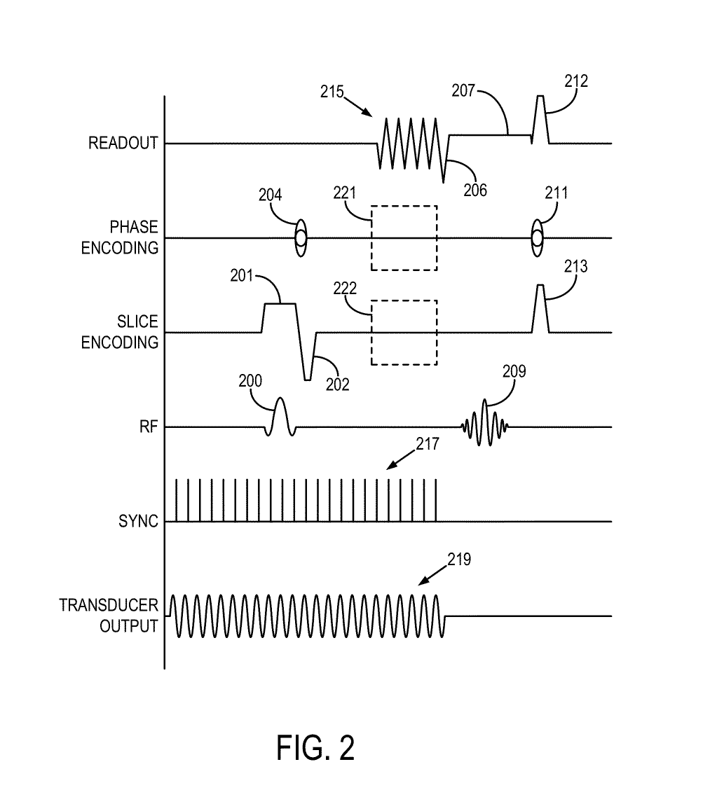

Referring particularly to FIG. 2, an exemplary pulse sequence, which may be used to acquire magnetic resonance (“MR”) data according to an embodiment of the present invention, is shown. The pulse sequence is fundamentally a 2DFT pulse sequence using a gradient recalled echo. Transverse magnetization is produced by a selective 90 degree radiofrequency (“RF”) excitation pulse 200 that is produced in the presence of a slice select gradient, G_{z}, pulse 201 and followed by a rephasing gradient pulse 202. A phase encoding gradient, G_{y}, pulse 204 is then applied at an amplitude and polarity determined by the view number of the acquisition. A read gradient, Gₓ, waveform is applied as a negative dephasing lobe 206, followed by a positive readout gradient pulse 207. An MR echo signal 209 is acquired 40 milliseconds after the RF excitation pulse 200 during the readout pulse 207 to frequency encode the 256 digitized samples. The pulse sequence is concluded with spoiler gradient pulses 212 and 213 along read and slice select axes, and a rephasing gradient pulse 211 is applied along the phase encoding axis (“G_{y}-axis”). As is well known in the art, this rephasing pulse 211 has the same size and shape, but opposite polarity of the phase encoding pulse 204. The pulse sequence is repeated 128 times with the phase encoding pulse 204 stepped through its successive values to acquire a 128-by-256 array of complex MR signal samples that comprise the data set.

An alternating magnetic field gradient is applied after the transverse magnetization is produced and before the MR signal is acquired. In the pulse sequence illustrated in FIG. 2, the read gradient, Gₓ, is used for this function and is alternated in polarity to produce bipolar, gradient waveforms 215. The frequency of the alternating gradient 215 is set to the same frequency used to drive the MRE transducer, and it typically has a duration of 25 milliseconds. At the same time, the pulse sequence server 110 produces synchronizing (“sync”) pulses 217, which have the same frequency as and have a specific phase relationship with respect to the alternating gradient pulses 215. These sync pulses 217 are used to produce the drive signals for the magnetic resonance elastography (“MRE”) transducer to apply an oscillating stress 219 to the patient. To insure that the resulting waves have time to propagate throughout the field of view, the sync pulses 217 may be turned on well before the pulse sequence begins, as shown in FIG. 2.

The phase of the MR signal 209 is indicative of the movement of the spins. If the spins are stationary, the phase of the MR signal is not altered by the alternating gradient pulses 215, whereas spins moving along the read gradient axis (“Gₓ-axis”) will accumulate a phase proportional to their velocity. Spins which move in synchronism and in phase with the alternating magnetic field gradient 215 will accumulate maximum phase of one polarity, and those which move in synchronism, but 180 degrees out of phase with the alternating magnetic field gradient 215 will accumulate maximum phase of the opposite polarity. The phase of the acquired MR signal 209 is thus affected by the “synchronous” movement of spins along the Gₓ-axis.

The pulse sequence in FIG. 2 can be modified to measure synchronous spin movement along the other gradient axes (G_{y} and G_{z}). For example, the alternating magnetic field gradient pulses may be applied along the phase encoding axis (“G_{y}-axis”) as indicated by dashed lines 221, or they may be applied along the slice select axis (“G_{z}-axis”) as indicated by dashed lines 222. Indeed, they may be applied simultaneously to two or three of the gradient field directions to “read” synchronous spin movements along any desired direction.

MRE may be implemented using most types of MR imaging pulse sequences. Gradient echo sequences can be readily modified to incorporate the alternating gradient as illustrated in the above-described embodiment. In some cases, however, the characteristics of a gradient echo sequence may not be ideal for a particular application of the technique. For example, some tissues (such as those with many interfaces between materials with dissimilar magnetic susceptibilities) may have a relatively short T*₂ relaxation time and, therefore, may not provide enough signal at the required echo delay time. In this setting, a spin echo implementation of the invention may be ideal, because for a given echo delay time (“TE”), this pulse sequence is much less sensitive to susceptibility effects than a gradient echo sequence. When a spin echo pulse sequence is used, the alternating magnetic field gradient can be applied either before and/or after the 180 degree RF inversion pulse. However, if the alternating gradient is applied both before and after the RF inversion pulse, the phase of the alternating magnetic field gradient must be inverted 180 degrees after the RF inversion pulse in order to properly accumulate phase.

The physical properties of tissue are measured using MRE by applying a stress and observing the resulting strain. For example a tension, pressure, or shear is applied to a subject and the resulting elongation, compression, or rotation is observed. By measuring the resulting strain, elastic properties of the tissue such as Young's modulus, Poisson's ratio, shear modulus, and bulk modulus can be calculated. Moreover, by applying the stress in all three dimensions and measuring the resulting strain, the elastic properties of the tissue can be completely defined.

The attenuation of the strain wave can be estimated by observing the rate at which the strain decreases as a function of distance from the stress producing source. From this, the viscous properties of the gyromagnetic medium may be estimated. The dispersion characteristics of the medium can be estimated by observing the speed and attenuation of the strain waves as a function of their frequency. Dispersion is potentially a very important parameter for characterizing tissues in medical imaging applications.

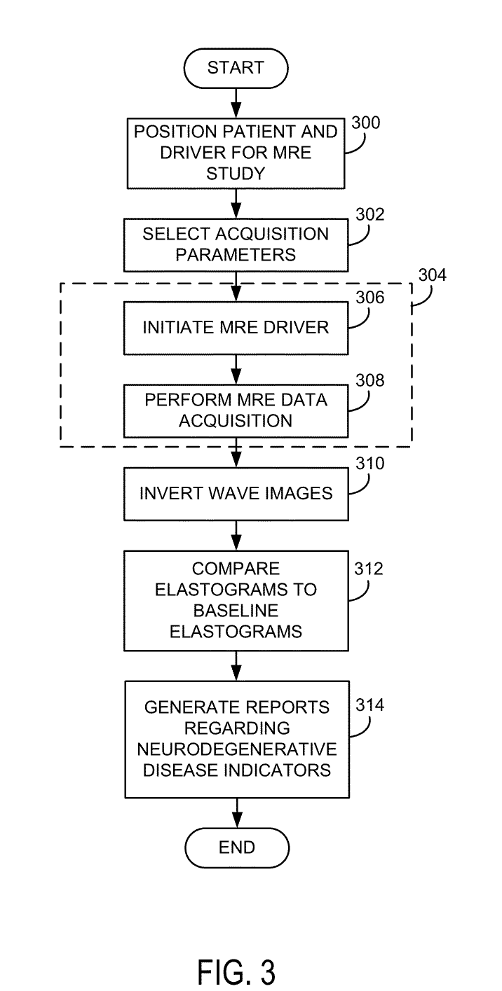

Referring to FIG. 3, a flow chart is provided setting forth the steps of a method for performing brain MRE, which may include three-dimensional (3D) MRE, using a soft pillow driver and a single-shot spin-echo EPI pulse sequence. As will be described, using the present invention, it can be seen that patients with AD have significantly softer brain parenchyma than patients without AD.

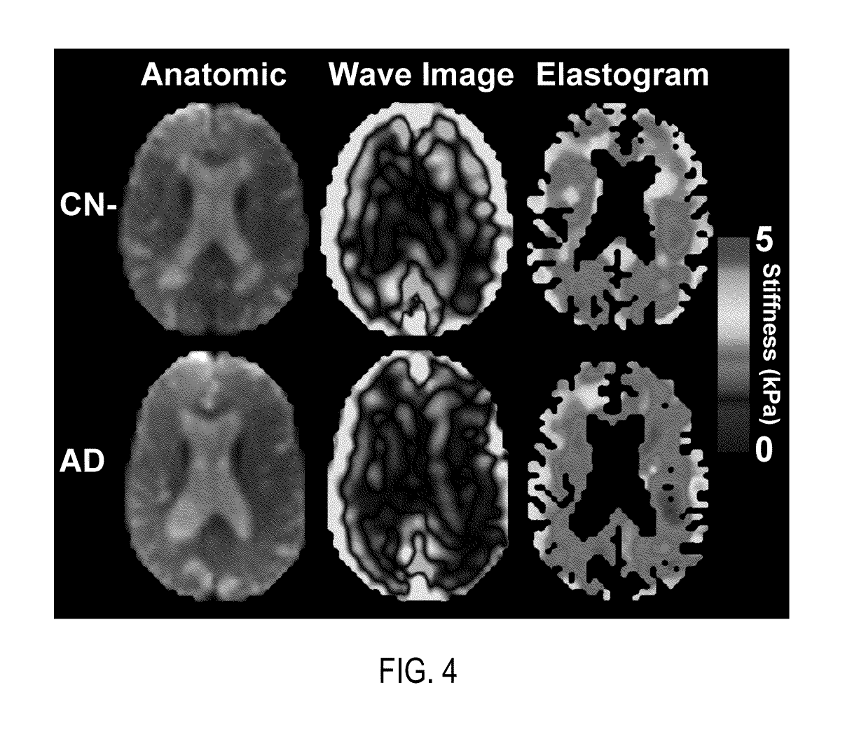

The process starts by positioning the patient in an MRI system, such as described above, and arranging an MRE driver to make proper contact with the patient, as represented by process block 300. At process block 302, desired MRE acquisition parameters are selected. For example, the implementation of an accelerated spin-echo EPI MRE sequence at 3T allows for a fast acquisition of the 3D wave field. Thereafter, an MRE acquisition can begin, as generally indicated by block 304. However, since it is desirable that the methods of the present invention yield results that are readily reproducible, the abrupt transition from rest to full motion that could potentially lead to subject motion due to a startle response should be controlled. To deal with this concern, the driver may be initialized at process block 306, such as using a progressive ramping of the power from rest to full power, for example, over an 8 second period before data acquisition begins at process block 308. In addition, it may be desirable to select a vibrational frequency that is preferable to the subject. For example some tests concluded that a 60 Hz vibration was more comfortable than 55 Hz. Following data acquisition at process block 308, the acquired wave images are inverted at process block 310 and elastograms are generated. The algorithm utilized for calculating the curl of the wave data removes unwanted longitudinal and geometric wave effects, while also avoiding potential pitfalls of 3D phase unwrapping. The elastograms, as will be described, can be compared to baseline elastograms at process block 312 to then generate a report at process block 314, such as serves to illustrate or show in a clinically discernable mechanism, indicators of a neurodegenerative disease, such as AD. Specifically, the report or images provided yields a noninvasive measure of the change in brain stiffness due to brain amyloid load. For example, FIG. 4 shows anatomical MRI images, wave images, and elastograms for both normal or base line and AD groups.

In order to achieve the ability to illustrate or show in a clinically discernable mechanism, indicators of a neurodegenerative disease, such as AD, the present invention was based on new research and developments that provide a basis for such illustrations and/or the qualification and quantification of comparative metrics. Specifically, stiffness measurements utilizing the above-described techniques were conducted in healthy volunteers and found to be in agreement with current research literature regarding the stiffness of healthy brains. For example, some have reported a mean brain shear modulus of 1.56+1.07i kPa at 50 Hz in 6 normal volunteers with comparable reproducibility to the work presented here (Sack I, Beierbach B, Hamhaber U, Klatt D, Braun J. Non-invasive measurement of brain viscoelasticity using magnetic resonance elastography. NMR in Biomedicine 2008; 21:265-271). This complex shear modulus equates to a shear stiffness of 2.07 kPa. Although this work was done in two dimensions, a full evaluation of the experimental setup to demonstrate that a two-dimensional analysis should be sufficient to obtain an accurate stiffness inversion was completed. At a higher frequency of 90 Hz and using a 3D direct inversion algorithm, some reported that white matter had a shear modulus of 2.7+2.5i kPa for 5 volunteers, equating to a shear stiffness of 4.24 kPa (Green M A, Bilston L E, Sinkus R. In vivo brain viscoelastic properties measured by magnetic resonance elastography. NMR in Biomedicine 2008; 21:755-764). As expected, the average stiffness of the ten volunteers studied for purposes of the present invention, 3.07 kPa, lies between the work done at 50 and 90 Hz due to the dispersive and viscoelastic nature of brain tissue. Previous work by others, however, reported higher brain stiffness values than the work outlined above (Kruse S A, Rose G H, Glaser K J, et al. Magnetic Resonance Elastography of the Brain. Neuroimage 2008; 39(1):231-237). This discrepancy likely exists because this work was performed at a higher frequency (100 Hz) and only used a 2D acquisition and inversion that may not have been optimized to minimize through-plane wave propagation. Through-plane propagating waves can appear in 2D data as waves with longer wavelengths thus overestimating the stiffness of the tissue. The difference in stiffness observed between the 10 volunteers in the reproducibility study (3.07 kPa at 55 Hz) and the 14 CN− subjects (cognitively normal and absent significant amyloid load) in the AD study (2.37 kPa at 60 Hz) is expected due to their age difference (Sack I, Beierbach B, Wuerfel J, et al. The impact of aging and gender on brain viscoelasticity. Neuroimage 2009; 46(3):652-657). Accordingly, a baseline of healthy MRE elastogram information can be generated for use, for example, at process block 314 of FIG. 3.

Also, decreased brain stiffness was demonstrated with MRE in a group of AD patients compared to age- and gender-matched control subjects. The fact that patients were demented indicates that they were all at a stage in the Alzheimer's disease process where significant neurodegeneration had occurred, and that the entire AD pathological cascade had been engaged. Current thinking concerning AD pathogenesis is that the initial molecular events center on dysregulation of the processing of amyloid precursor protein leading to an increase in production of amyloidogenic β-amyloid-42 (Aβ). Aβ oligomerizes to form toxic fibrils leading to formation of the amyloid plaques that are a pathological hallmark of the disease. Downstream pathological events include dysregulation of tau kinases leading to neurofibrillary tangles (the second pathological hallmark of AD), oxidative stress, and finally synaptic loss, cell death and dementia. Currently, biomarkers exist to assess Aβ load, such as Pittsburgh imaging compound B (PIB) or CSF Aβ42, neurofibrillary tangles (CSF tau), synaptic dysfunction (FDG PET), and neurodegeneration (structural MRI). The magnitude and rate of change of several biomarkers can be combined to determine the grade of an individual's disease.

Throughout an AD cascade, several processes may impact the mechanical properties of brain parenchyma. The amyloid fibrils themselves are six orders of magnitude greater in stiffness than neurons and glia (Lu Y B, Franze K, Seifert G, et al. Viscoelastic properties of individual glial cells and neurons in the CNS. PNAS 2006; 103(47):17759-17764 and Smith J F, Knowles T P, Dobson C M, MacPhee C E, Welland M E. Characterization of the nanoscale properties of individual amyloid fibrils. PNAS 2006; 103(43):15806-15811). As a result, it is counterintuitive that global brain stiffness might decrease due to AD and/or be sufficiently changed to serve as a biomarker, particularly, for non-invasive, imaging. One hypothesis was that the aggregation of these stiff proteins containing β-pleated sheets would lead to an increase in the global brain stiffness. Not only did the AD group demonstrate decreased brain stiffness, but the CN+ group (cognitively normal with significant amyloid load) was not different from the CN− group indicating that the presence of brain amyloid alone is not responsible for the observed change in brain stiffness. On the other hand, the decrease in stiffness may reflect a host of microstructural events that destroy normal cytoarchitectural integrity, such as degradation of the extracellular matrix following the deposition of hydrophobic amyloid protein, cytoskeletal disruption downstream of tau hyperphosphorylation, and loss of the interconnecting synaptic networks. Attributing the MRE findings to disease-related loss of microstructural integrity is consistent with well-established findings of increased mean diffusivity and decreased fractional anisotropy in AD on diffusion imaging. For example, FIG. 4 illustrates that the loss of brain stiffness in AD relative to CN is not limited to the cortex, which is where amyloid plaques are located, but also involves white matter. To this end, it is contemplated that data may be acquired, such as diffusion tensor or diffusion spectrum imaging data, to create fiber tracts in the brain (white matter tracts) that can be correlated and oriented with stiffness and MRE information.

It is important to note that PIB, though a well-established amyloid PET tracer used to establish the presence or absence of brain amyloid when studying Alzheimer's disease, was revealed by the present invention to have substantial limitations, for example, when making clinical diagnosis and performing clinical analysis. Specifically, the present invention, when tested using the following testing protocol, detected a significant decrease in stiffness in subjects who were indicated by analysis based only on PIB-based protocols as having AD (PIB-positive) and were otherwise verified as having Alzheimer's disease. However, the present invention was able to additionally distinguish subjects that were determined to be PIB-positive, yet otherwise tested as cognitively normal, compared to PIB-positive AD subjects. This was achieved through an indication of substantially “no change in stiffness” for the PIB-positive and cognitively normal subjects.

It is noted that a L/R occipital asymmetry in the elastograms was observed that is not a function of driver orientation, but may be based on anatomy or anatomy's impact on inversion. Also, the stiffness of temporal/parietal lobes has potential to differentiate PIB+ and PIB− controls using regional analysis of AD data.

That is, the present invention recognizes that, since diseases of the brain have characteristic topographies, MRE can be used as a tool to measure regional stiffness. Specifically, regional repeatability in addition to that of global stiffness can be considered. To do so, a T1 template along with a labeled lobar atlas can be warped to each individual's T1-weighted image using a unified segmentation algorithm. The T1 image is segmented to calculate gray matter (GM), white matter (WM) and cerebrospinal fluid (CSF) content at each voxel. The segmented images along with the warped atlas are then registered and resliced to the magnitude image of the MRE data using the T1 image as the reference image so the GM, WM, and CSF content, as well as the atlas region, is known for each voxel of the MRE data.

So-called adaptive methods allow for a further refinement of regional stiffness estimation. In the initial pipeline for regional stiffness measurement, the stiffness map can be calculated using displacement data from the entire brain and then a stiffness map can be parceled into different ROIs based on the brain atlas. The downside to this approach is that the stiffness map is, in effect, a low pass filtered image of the true stiffness, so stiffness in one region will impact the stiffness calculated in any adjacent region. Using the adaptive methods, the displacement images can be masked as the very first step, then a unique stiffness map can be calculated for each ROI knowing that its stiffness was computed without contributions from adjacent brain tissue. Adaptive methods make this pipeline possible because a single erosion from each ROI leaves a sufficient number of voxels to calculate a stable estimate of the regional stiffness, but three erosions would substantially, and potentially overly reduce the number of voxels. Thus, this method creates regional elastograms from an eroded model rather than eroding a global elastogram. This pipeline improves repeatability.

When measuring global brain stiffness with the adaptive methods, the AD and CN groups demonstrate a highly significant difference (p=0.0057, Wilcoxon rank sum). The voxels nearest to the brain's edge contribute most to AD and CN discrimination, and using methods in accordance with the present invention, the vast majority of these voxels can be saved without introducing edge-related bias. Studies using the present invention fit the known topography of AD, indicating significant decreases in brain stiffness in the frontal, parietal, and temporal lobes. These lobes that contain association cortices are hardest hit by AD. Conversely brain regions that contain primary cortices, namely the occipital lobes and the sensory/motor strips, show no significant group-wise differences. Likewise the cerebellum shows no significant change in stiffness related to AD. Based on such results, the ROI can be tailored to include the frontal, parietal, and temporal lobes excluding the sensory/motor strip. Such an ROI was shown to outperform global brain stiffness (p=0.003, Wilcoxon rank sum).

In addition to the determination of decreased brain stiffness due to AD presented herein, decreased brain stiffness due to multiple sclerosis and in patients with normal pressure hydrocephalus have been reported. While these results may suggest that MRE is an unspecific exam, it does have potential to improve the sensitivity of diagnosis for several diseases when used in the context of a patient's clinical background. For example, prolonged T2 relaxation is often an unspecific finding in a pathological process, yet it is a useful MR feature in diagnosing many neurological diseases.

### Example

The above-described systems and methods were experimentally tested to investigate patient acceptance and reproducibility of the 3D MRE brain exam using the soft vibration source, and to determine if MRE could noninvasively measure a change in the elastic properties of the brain parenchyma due to AD within clinical constraints.

Subjects were identified, recruited and imaged using the above-described systems and methods. To test the technique's reproducibility, ten male volunteers all without known neurological diseases were recruited with a median age of 29 years (range from 25 to 52 years). MRE was performed on each individual a total of 4 times in 2 sessions separated by an average of 8.7 days (range from 4 to 20 days). On each day, a complete MRE exam was performed and then the patient removed from the MRI table and the actuator components were disassembled. Subsequently the equipment and subject were again positioned on the MRI table and a second complete MRE exam was performed.

To examine the effect of AD on brain stiffness, 28 subjects were recruited including 7 with probable AD, 14 age- and gender-matched PIB-negative cognitively normal controls (CN−) and 7 age- and gender-matched PIB-positive cognitively normal controls (CN+). All subjects were identified from The Mayo Clinic Study of Aging (MCSA) and Alzheimer's Disease Patient Registry (ADPR) data base. All subjects recruited into the ADRC and ADPR are followed prospectively. Criteria for the diagnosis of cognitively normal controls were: 1) no active neurologic or psychiatric disorders, 2) any ongoing medical problems or their treatments did not interfere with cognitive function, 3) a normal neurological exam, 4) no psychoactive medications, and 5) were independently functioning community dwellers. The diagnosis of probable AD was made according to the Diagnostic and Statistical Manual for Mental Disorders, III Edition-Revised (DSM-III-R) Criteria for dementia, and National Institute of Neurological and Communicative Disorders and Stroke/Alzheimer's Disease and Related Disorders Association Criteria (NINCDS/ADRDA) for AD. Pittsburgh Compound B (PIB) is the most widely studied PET amyloid imaging ligand to date. Approximately ⅓ of cognitively normal elderly subjects harbor a significant amyloid plaque load, one of the cardinal pathological features of AD. As part of the MCSA and ADRC studies, all subjects had already undergone brain amyloid imaging with PIB to establish the presence or absence of Aβ brain amyloid. Subjects with a global cortical PIB score of less than 1.5 (the ratio of uptake in the cortex versus a cerebellar reference ROI) were considered PIB-negative, while scores above 1.5 were considered PIB-positive. The median age of the CN− group was 81.5 (range: 75-89), the median age of the CN+ group was 83 (range: 73-93), and the median age of the AD group was 85 (range: 76-94) (p=0.17, Kruskal-Wallis). The CN− group consisted of 10 men and 4 women while the CN+ and AD groups consisted of 5 men and 2 women.

MRE data was collected with a single-shot spin-echo EPI pulse sequence on a 3.0T MR imager (SIGNA Excite, GE Healthcare, Waukesha, Wis.). Shear waves were introduced into the brain through a soft pillow-like vibration source using a pneumatic actuator. The active component of the actuator, located outside of the scan room, was comprised of a waveform generator, an amplifier, and an acoustic speaker. The passive pillow-like component consisted of a soft, inelastic, fabric cover over a porous, springy, mesh measuring 15×9×1.5 cm. The soft vibration source was placed under the subject's head within an 8 channel receive-only head coil. The active and passive driver components were connected by a 24-foot long, 0.75-inch diameter flexible tube from the active driver terminating in a 0.5-inch diameter, 1.5-foot long tube integrated into the passive driver.

For the reproducibility experiments, the driver system was operated at 55 Hz and the resulting tissue motion was imaged with the EPI MRE imaging sequence using the following parameters: axial slices, TR/TE=1636/64.0 ms, FOV=25.6 cm, BW=±250 kHz, 60×60 imaging matrix reconstructed to 64×64, 3×ASSET acceleration, 2.5-mm thick slices with a 1.5-mm skip, one 4-G/cm 18.2-ms zeroth- and first-order moment nulled motion-encoding gradient on each side of the refocusing RF pulse synchronized to the motion, motion encoding in the positive and negative x, y and z directions, and 4 phase offsets sampled over one period of the 55-Hz motion.

For the study of AD, the driver system was operated at 60 Hz and the resulting tissue motion was imaged with the EPI MRE imaging sequence using the following parameters: axial slices, TR/TE=1500/61.3 ms, FOV=25.6 cm, BW=±250 kHz, 60×60 imaging matrix reconstructed to 64×64, 3×ASSET acceleration, 2.5-mm thick slices with a 1.5-mm skip, one 4-G/cm 18.2-ms zeroth- and first-order moment nulled motion-encoding gradient on each side of the refocusing RF pulse synchronized to the motion, motion encoding in the positive and negative x, y and z directions, and 4 phase offsets sampled over one period of the 60-Hz motion. A second MRE scan of approximately 1.5 minutes was performed with the motion source turned off sampling only 2 phase offsets 90° apart to provide additional data for the signal-to-noise ratio (SNR) calculations. The resulting images had isotropic 4-mm resolution and required at most a 3.5-minute total acquisition time.

Eighteen slices covering the cerebrum were used for image processing in all subjects. The first temporal harmonic of the vector curl of the wave data was calculated from the phase images to remove contributions from longitudinal wave propagation and static phase errors. The spatial derivatives were calculated using central differences over a 3×3×3 window. The first-harmonic curl data were prefiltered with a 3×3×3 filter of the form (1−x²)²(1−y²)²(1−z²)² where −1≦x, y, z≦1, and inverted with a 3D direct inversion (DI) algorithm. The median stiffness for each individual was reported from a global region of interest (ROI). For display purposes, the elastograms were filtered with a 3×3×3 median filter.

For the reproducibility experiments, the ROI used for reporting the median tissue stiffness included the whole brain excluding 3 voxels from the edge of the calvarium (approximately ⅓ of the shear wavelength), the longitudinal fissure, the ventricles and low magnitude signal regions such as the midbrain.

For the AD experiments, the curl calculation as described above was also performed on the data acquired with the motion turned off. The standard deviation of the no-motion curl data in 3×3×3 sliding windows was used as an estimate of the local noise of the curl data. The local SNR of the curl data was calculated as the pixel-by-pixel ratio of the amplitude of the first-harmonic of the curl data to the noise standard deviation. The ROI utilized to calculate the tissue stiffness included the portion of the brain with SNR>5, that was at least 3 voxels from the brain surface and the longitudinal fissure to remove edge artifacts, and excluded any voxels with a cerebrospinal fluid (CSF) content greater than 30%. The edge voxels were removed by first thresholding the magnitude images to create a mask of the brain, then manually drawing a line along the longitudinal fissure to create a line of voxels that were removed from all slices, and finally 3 serial erosions with a 3×3×3 structural element. CSF content was calculated by segmenting a 3D T1 weighted image as described by Jack C R, Lowe V J, Senjem M L, et al. ¹¹C PiB and structural MRI provide complimentary information in imaging of Alzheimer's disease and amnestic mild cognitive impairment. Brain 2008; 131:665-680. The magnitude data from the MRE acquisition were registered to the T1 images with a rigid body transformation, and the segmented CSF images were resliced to the MRE data to obtain the CSF content for each voxel of the MRE data.

The CN−, CN+ and AD groups were compared using the Kruskal-Wallis one-way analysis of variance. The Wilcoxon rank sum test was used for pair wise comparisons of the groups to determine which were significantly different from one another.

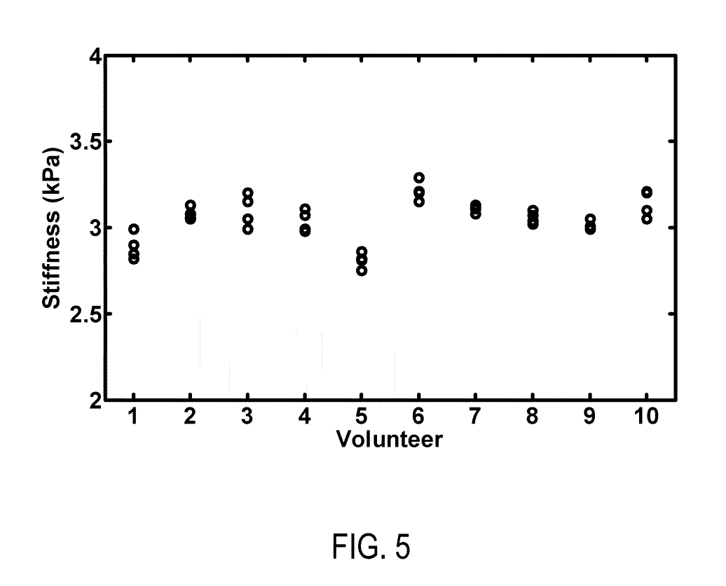

All 40 reproducibility exams were completed successfully and provided data adequate for inversion. The median stiffness for the ten volunteers was 3.07 kPa (interindividual range: 2.81-3.21 kPa). The results indicated that 3D brain MRE can be performed with a coefficient of variation of 3.1% or less. Summary data for each subject are shown in FIG. 5. Note the tight distribution of stiffness measurements for each individual. The median and maximum coefficients of variation were 1.71% and 3.07%, respectively.

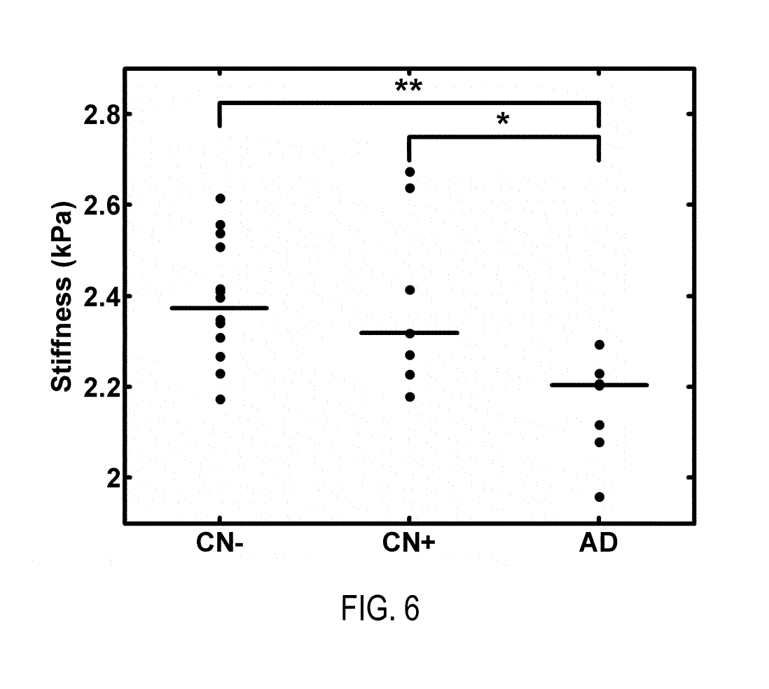

In the AD study, MRE demonstrated a significant difference in the brain stiffness of AD subjects compared to age- and gender-matched controls, such as illustrated in FIG. 6. The median stiffness of the CN− group was 2.37 kPa (range: 2.17-2.62 kPa, n=14), the median stiffness of the CN+ group was 2.32 kPa (range: 2.18-2.67 kPa, n=7), and the median stiffness of the AD group was 2.20 kPa (range: 1.96-2.29 kPa, n=7) (p=0.0055, Kruskal-Wallis). Pair wise comparisons with the Wilcoxon rank sum test indicated that both the CN− group (p=0.0015) and the CN+ group (p=0.026) were significantly different from the AD group. The CN− and CN+ groups did not differ from each other (p=0.85). Example magnitude images, wave images and stiffness maps from an AD subject and an age-matched CN− subject are shown in FIG. 4.

These results demonstrate that MRE can noninvasively measure changes in the mechanical properties of the human brain due to AD (FIG. 6). The average CSF content within the ROI of each subject did not differ between the three groups (p=0.62, Kruskal-Wallis) and was less than 2% of the ROI on average, ensuring that the stiffness change results from a change in the mechanical properties of the brain parenchyma and is not a reflection of CSF volume change due to atrophy.

Thus, the present invention provides a system and method for 3D brain MRE that can be performed reproducibly based on a discovery and proof that AD pathology alters the mechanical properties of brain in a way that can be measured in vivo by MRE. Measures of brain elasticity should provide unique insights into fundamental ultrastructural alterations of the brain that occur in AD, as well as how these change with time, correlate with other disease biomarkers and with clinical expression of the disease.

In particular, amyloid is the earliest hallmark pathology of Alzheimer's disease. PIB, though a well-established amyloid PET tracer used to establish the presence or absence of brain amyloid when studying Alzheimer's disease, was revealed by the present invention to have substantial limitations, for example, when making clinical diagnosis and performing clinical analysis that are overcome by the present invention. Specifically, the present invention, when tested using the following testing protocol, detected a significant decrease in stiffness in subjects who were indicated by analysis based only on PIB-based protocols as having AD (PIB-positive) and were otherwise verified as having Alzheimer's disease. However, the present invention was able to additionally distinguish subjects that were determined to be PIB-positive, yet otherwise tested as cognitively normal, compared to PIB-positive AD subjects. This was achieved through an indication of substantially “no change in stiffness” for the PIB-positive and cognitively normal subjects.

The present invention is employed in a system such as that described in the previously-cited U.S. Pat. No. 5,592,085 which provides a system and method for measuring the strain in gyromagnetic materials, such as tissues, using MR methods and apparatus and is incorporated herein by reference. The present invention may also be employed with other medical imaging modalities including, but not limited to, ultrasound.

The present invention produces and delivers stress levels that are much larger than those produced by prior art drivers, even other passive acoustic drivers. Unlike the prior art passive drivers which have a rigid housing a diaphragm mounted thereon, the embodiments of the present invention closely and comfortably couples to the subject for consistent driver efficiency and imaging.

1. A method for generating a report on a subject using data acquired using a magnetic resonance imaging (MRI) system:
positioning a subject within the MRI system;
coupling a driver to the subject to impart vibrational energy to the subject;
using the MRI system and in coordination with operation of the driver, acquiring medical imaging data from the subject's brain;
deriving stiffness information of the subject's brain from the medical imaging data; and
generating a report indicating the stiffness information of the subject's brain relative to at least one of baseline stiffness information and amyloid tracer information to indicate a status of the subject with respect to a neurodegenerative disease.

2. The method of claim 1 wherein acquiring medical imaging data includes reconstructing an elastogram of the subject's brain and generating the report includes comparing the elastogram of the subject's brain to an elastogram of a baseline subject's brain.

3. The method of claim 2 wherein generating the report includes providing the elastogram of the subject's brain and the elastogram of the baseline subject's brain and wherein the elastogram of the subject's brain and the elastogram of the baseline subject's brain are color coded to reflect relative stiffness information to indicate the status of the subject with respect to the neurodegenerative disease.

4. The method of claim 1 wherein generating the report includes indicating a decrease in the stiffness information of the subject's brain when compared to the baseline stiffness information corresponds to a relative increase in a likelihood of the subject having the neurodegenerative disease.

5. The method of claim 1 wherein generating the report includes indicating a consistency in the stiffness information of the subject's brain when compared to the baseline stiffness information corresponds to a relative non-change in a likelihood of the subject having the neurodegenerative disease.

6. The method of claim 1 wherein the baseline stiffness information includes medical imaging data acquired from the subject during a prior medical imaging process.

7. The method of claim 1 wherein the baseline stiffness information includes medical imaging data acquired from subjects known to have the neurodegenerative disease and subject known to be cognitively normal.

8. The method of claim 1 wherein generating the report includes indicating an elastogram of the subject's brain.

9. The method of claim 1 wherein the neurodegenerative disease is Alzheimer's disease.

10. The method of claim 1 wherein the amyloid tracer information includes Pittsburgh imaging compound B (PIB) information.

11. A magnetic resonance imaging (MRI) system comprising:
a magnet system configured to generate a polarizing magnetic field about at least a portion of a subject;
a plurality of gradient coils configured to apply a gradient field to the polarizing magnetic field;
a radio frequency (RF) system configured to apply an excitation field to the subject and acquire MR image data therefrom;
a driver system configured to deliver an oscillatory stress to the subject to, thereby, direct a shear wave through the subject;
a computer system programmed to:
control operation of the gradient coils and the driver system to coordinate characteristics of the oscillatory stress with application of the gradient field;
control operation of the RF system to acquire medical imaging data from the subject's brain;
determine a plurality of regions of interest (ROIs) within the subject's brain;
derive stiffness information of the subject's brain from the medical imaging data by determining a stiffness map for each ROI without contributions from brain tissue adjacent to the ROI; and
generate an image of the subject's brain from the medical imaging data indicating a status of the subject with respect to a neurodegenerative disease, based on the stiffness map for each ROI.

12. The system of claim 11 wherein the computer system is further programmed to generate the image to indicate a relative stiffness of the subject's brain with respect to baseline stiffness information to indicate the status of the subject with respect to the neurodegenerative disease.

13. The system of claim 11 wherein the computer system is further programmed to reconstruct an elastogram of the subject's brain to derive the stiffness information and compare the elastogram of the subject's brain to an elastogram of a baseline subject's brain to indicate the status of the subject with respect to the neurodegenerative disease.

14. The system of claim 11 wherein the computer system is further programmed to determine a decrease in the stiffness information of the subject's brain when compared to baseline stiffness information and, based thereon, indicate an increase in a likelihood of the subject having the neurodegenerative disease in the image.

15. The system of claim 11 wherein the computer system is further programmed to determine a consistency in the stiffness information of the subject's brain when compared to baseline stiffness information and, based thereon, indicate a relative non-change in a likelihood of the subject having the neurodegenerative disease.

16. The system of claim 11 wherein the computer system is further programmed to determine a global brain stiffness decrease and correlate the global decrease in brain stiffness to an adverse pathological condition.

17. The system of claim 16 wherein the adverse pathological condition may include Alzheimer's disease.

18. The system of claim 11 wherein the computer is further programmed to derive fiber tract information from the medical imaging data and correlate therewith the image of the subject's brain indicating the status of the subject with respect to the neurodegenerative disease, based on the stiffness information.

19. The system of claim 11 wherein determining the stiffness map for each ROI without contributions from brain tissue adjacent to the ROI includes performing a single erosion from each ROI.
